(19) United States
(12) Patent Application Publication
(10) Pub. No.: US 2025/0299827 A1
(43) Pub. Date: Sep. 25, 2025

(54) ESTABLISHMENT AND USE OF INDEX FOR EVALUATING MALIGNANCY LEVEL OF TISSUE MICROENVIRONMENT

(71) Applicant: National Taiwan University, Taipei City (TW)

(72) Inventors: Sheng-Fang SU, Taipei City (TW); Chao-Chi HO, Taipei City (TW); Huei-Wen CHEN, Taipei City (TW); Hao HO, Taipei City (TW); Ker-Chau LI, Taipei City (TW)

(21) Appl. No.: 18/863,224

(22) PCT Filed: Jul. 6, 2022
(86) PCT No.: PCT/CN2022/104183

§ 371 Date: Nov. 5, 2024

(51) Int. Cl. G16H 50/30; G16B 25/10

(52) U.S. Cl. CPC G16H 50/30; G16B 25/10

(57) ABSTRACT

The present disclosure provides an establishment and use of index for evaluating malignancy level of tissue microenvironment, which can be used to detect the potential of an individual to develop precancerous lesions and to predict the recurrence of tumors without relying on patient survival data.

### FIELD OF THE INVENTION

The present invention relates to establishment and use of index for evaluating tissue microenvironment, and in particular to establishment and use of index for evaluating malignancy level of tissue microenvironment.

### THE PRIOR ARTS

The multistep process of tumor development accompanies the coevolution of the tumor microenvironment (TME) that engages critical participants during tumorigenesis through direct and indirect interaction with tumors. The longstanding seed and soil theory highlights the TME in tumor metastasis and many aspects of oncogenic behaviors. Located in the vicinity of the tumors, cancer-associated fibroblasts (CAFs) possess multifaceted capacity for supporting tumor growth both in vitro and in vivo, promoting cancer cell migration and metastasis possibly through traveling with circulating tumor cells, remodeling the extracellular matrix (ECM), and being involved in angiogenesis, inflammation, immunosuppressive, and CAF-tumor reciprocal crosstalk. CAFs maintain cancer stemness via paracrine signaling communication with cancer stem cells, contributing to tumor recurrence, drug resistance, and immune response modulation.

CAFs exhibit differential gene expression patterns from their normal counterparts, and genome-wide analysis has also identified prognostic signatures in non-small-cell lung cancer (NSCLC) and breast cancer. Global DNA hypomethylation and promoter hypermethylation in tumor suppressor genes have been used as prognostic markers and were found to correlate with altered regulation of gene expression. Although the role of aberrant DNA methylation has been much appreciated in cancer, global profiling of DNA methylation in CAFs simultaneously characterized with gene expression has been less comprehensive.

### SUMMARY OF THE INVENTION

A primary objective of the present invention is to provide a method of establishing index for evaluating malignancy level of tissue microenvironment, comprising: (a) analyzing cancer-associated fibroblasts and normal fibroblasts from a cancer patient to obtain differentially methylated CpG sites and differentially expressed genes; (b) overlapping the differentially methylated CpG sites with the differentially expressed genes to obtain a differentially methylated-differentially expressed pair; (c) analyzing correlation between DNA methylation change value and gene expression fold change in the differentially methylated-differentially expressed pair to obtain a differentially methylated site with cis-correlation; (d) analyzing another differentially methylated site associated with clinical characteristics of the cancer patient and intersecting with the differentially methylated site to obtain a plurality of target sites; (e) performing principal component analysis on a group consisting of methylation β values of the plurality of target sites; (f) obtaining a loading value of a first principal component analysis of the methylation β values corresponding to each of the plurality of target sites; and (g) multiplying the methylation β values of the plurality of target sites by the loading value and summed to establish the index for evaluating malignancy level of tissue microenvironment.

According to an embodiment of the present invention, the cancer patient is a lung cancer patient, and one of the clinical characteristics is smoking.

Another objective of the present invention is to provide a method for evaluating malignancy level of tissue microenvironment, comprising: (a) determining a methylation β value of a target cell at a plurality of target sites in an in vitro sample of a subject; (b) multiplying the methylation β value of the plurality of target sites by a loading value of corresponding target site in the subject and summed to obtain an index score for evaluating malignancy level of tissue microenvironment; and (c) determining the tissue microenvironment in the subject to have potential to promote tumor malignancy when the index score is higher than a cutoff point, wherein the loading value of corresponding target site in the subject is a loading value of a first principal component analysis obtained by performing principal component analysis on a group, wherein the group consists of the methylation β value of the plurality of target sites in the target cell of a subject suffering from tumor.

According to an embodiment of the present invention, the subject suffering from tumor is a subject suffering from lung cancer.

Another objective of the present invention is to provide a system for evaluating malignancy level of tissue microenvironment, comprising: a detection module configured to determine and obtain a methylation β value of a target cell at a plurality of target sites in an in vitro sample of a subject; an analyzing and processing module configured to multiply the methylation β value of the plurality of target sites by a loading value of corresponding target site in the subject and summed to obtain an index score for evaluating malignancy level of tissue microenvironment and predicting tumor recurrence and precancerous lesions; and an information comparison module configured to compare whether the index score is higher than a cutoff point to determine whether the tissue microenvironment in the subject has or does not have potential to promote tumor malignancy.

According to an embodiment of the present invention, the potential to promote tumor malignancy is potential to produce precancerous lesions and/or to promote cancer recurrence.

The present invention has established index for evaluating malignancy level of tissue microenvironment. A methylation index that summarizes the β values of these CpGs was built for NF/CAF discrimination (MIND) with high sensitivity and specificity. The potential of MIND in detecting premalignancy across individual patients was shown in the present invention. MIND succeeded in predicting tumor recurrence in multiple lung cancer cohorts without reliance on patient survival data, suggesting that the malignancy level of TME may be effectively graded by this index. Precision TME grading may provide additional pathological information to guide cancer prognosis and open up more options in personalized medicine.

In order to enable those with ordinary skills in the art to understand the purpose, features, and effects of the present invention, the present invention is now described in detail through the following specific embodiments and in conjunction with the accompanying drawings.

### BRIEF DESCRIPTION OF DRAWINGS

FIG. 1 shows the schematic flow chart of the method of establishing MIND of the present invention.

FIG. 2 shows treating lung cancer cells with conditioned medium (CM) from paired CAFs and NFs to examine their contributions to the tumor-promoting ability in cancer migration, invasion, and sphere formation.

FIG. 3 shows volcano plot of the differentially expressed (DE) probes between CAFs and NFs. A probe is significant if the differential expression >1.5 fold and the false discovery rate control Q<0.1.

FIG. 4 shows analysis of differential gene expression between CAFs and NFs. Analysis of genes, upregulated or downregulated (n=19 or n=9 per gene). Actin was used as the internal control. Analysis was performed with Mann-Whitney U test. Data presented as mean±SD. Box plots display the median, first and third quartiles with whiskers as maximum and minimum values, and wherein symbols represent individual samples. * P<0.05, ** P<0.01.

FIG. 5 shows that using DAVID analysis, physiological pathways were significantly enriched in the differentially expressed genes at the false discovery rate of 0.1.

FIG. 6 shows the UpSet plot for the intersections among the tumor histology, stage, age, sex, and smoking status of the differentially expressed probes.

FIG. 7 shows volcano plot of the differentially methylated (DM) CpG sites between CAFs and NFs. The CpG site is significant if the difference in methylation β value is greater than 0.1 and the false discovery rate control Q is less than 0.1.

FIG. 8 shows hypermethylated and hypomethylated genes in differential methylation of CAF and NF (n=8-10 per gene). Analysis was performed with Mann-Whitney U test. Data presented as mean±SD. Box plots display the median, first and third quartiles with whiskers as maximum and minimum values, and wherein symbols represent individual samples. * P<0.05, ** P<0.01, *** P<0.001, **** P<0.0001.

FIG. 9 shows the UpSet plot for the intersections among the tumor histology, stage, age, sex, and smoking status of the differentially methylated CpG sites.

FIG. 10 shows the mean B-value difference in DNA methylation versus the mean log 2 fold change in gene expression of the 1193 DM-DE pairs. The gray zone reflects the selective threshold.

FIG. 11 shows hierarchical DNA methylation clustering of the 52 CAF and NF samples from 26 patients. Unmethylated is indicated in blue, methylated is in yellow. Principal component analysis (PCA) was performed and the loading of the first component (PC1) is shown on the right. The expression profile of the matched genes is also shown (bottom). Rows represent probes and columns represent samples. Clinical parameters including relapse are indicated in the bottom.

FIG. 12 shows scatter plots of DNA methylation versus gene expression in CAF and NF samples for target genes.

FIG. 13 shows the scatter plot of the first and second principal component (PC) analysis. Red, CAF; blue, NF. Triangles indicate samples from patients with relapse, while those with no relapse are shown as circles.

FIG. 14 shows the CAF/NF scores of the aforementioned 26 patients using MIND of the present invention, ordered from the largest value to the smallest value.

FIG. 15 shows the ROC curve evaluation of the performance of MIND of the present invention in discriminating CAF from NF in the aforementioned 26 patients.

FIG. 16 shows the ROC curve evaluation of the performance of MIND of the present invention in discriminating CAF from NF in another group of 14 non-small cell lung cancer patients.

FIG. 17 shows Kaplan-Meier analysis for relapse-free survival (RFS) prediction by MIND of the present invention in the 26 CAF samples.

FIG. 18 shows Kaplan-Meier analysis for relapse-free survival (RFS) prediction by MIND of the present invention in the 14 CAF samples.

FIGS. 19 and 20 show the significance of MIND of the present invention in predicting recurrence and patient survival rate in GSE39279 and TCGA-LUAD samples.

### DETAILED DESCRIPTION OF THE PREFERRED EMBODIMENT

All technical and scientific terms used in the context, unless otherwise defined, have meanings commonly understood by those with ordinary skills in the art.

The embodiments of the present invention would be further described below in conjunction with the accompanying drawings. The examples listed below are only used to illustrate the present invention and are not intended to limit the scope of the present invention. Any person with ordinary skills in the art can make some changes and modifications without departing from the spirit and scope of the present invention. Therefore, the scope of the present invention should be subject to the scope defined by the appended claims.

As used herein, the data provided represent experimental values that can vary within a range of ±20%, preferably within ±10%, and most preferably within ±5%.

### Materials and Methods

### Tissue Collection and Primary Culture System

We established a cell library of CAFs from surgically resected lung tumor tissues of non-small cell lung cancer (NSCLC) patients. The normal tissue counterparts were obtained at least 5 cm away from the neoplastic lesions. The primary cultures of lung CAFs and the paired NFs were processed as follows. The tissues were minced and digested with deoxyribonuclease I (BioShop) and collagenase I (Life Technologies) at 37° C. for 1 hour. After centrifugation, filtered cells were cultured in 6-well plates with Dulbecco's modified Eagle medium (DMEM) (Life Technologies) containing 10% FBS (Gibco) at 37° C. under 5% CO₂. In addition, corresponding conditioned medium (CM) from NFs and CAFs was carefully collected for the analysis of tumor-promoting capacity.

### Cell Lines

The human lung adenocarcinoma cell line A549 was purchased from American Type Culture Collection (ATCC, CCL-185). CL1-0 and CLS1 cells were cultured from lung cancer patients. The human bronchial epithelial cell line BEAS-2B was obtained from ATCC (CRL-9609).

The method for establishing the index for evaluating the malignancy level of the tissue microenvironment of the present invention, that is, establishing the distinct methylation index for NF/CAF discrimination (MIND) for discriminating NF from CAF; and the MIND of the present invention of the method for evaluating the malignancy level of the tissue microenvironment would be described in detail below.

### Example 1 Establishment of MIND of Present Invention

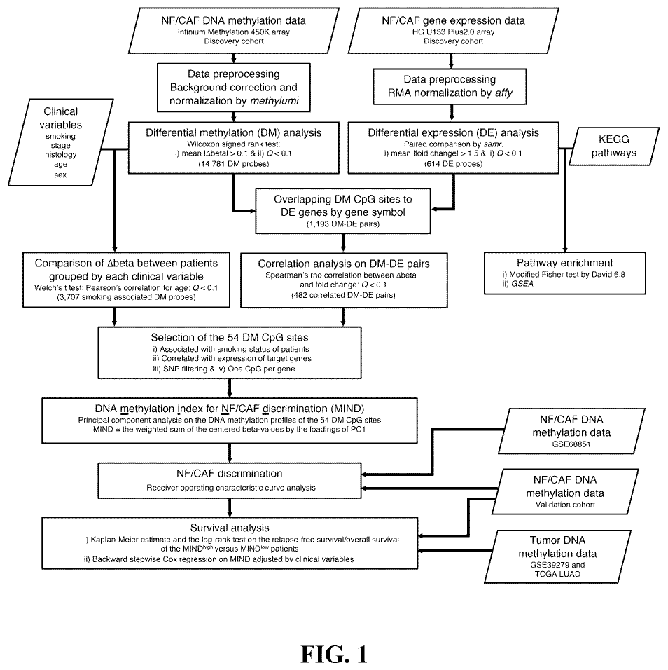

In the embodiment of the present invention, lung cancer patients would be taken as an example to illustrate the process and method of establishing the distinct methylation index for NF/CAF discrimination (MIND) of the present invention. As shown in FIG. 1, differential methylation (DM) of DNA was analyzed in cancer-associated fibroblasts (CAFs) and normal fibroblasts (NFs) of lung cancer patients, and differential expression (DE) of genes was analyzed. The targets of the two differential analyzes are overlapped to find the differential methylation-differential expression pair (DM-DE pair). The correlation between methylation changes and fold changes in expression within each DM-DE pair was evaluated to identify differentially methylated sites that are cis-related to the genes they encode. This site was intersected with the differentially methylated sites of DNA related to the clinical characteristics of lung cancer patients to select the target CpG site. Subsequently, the methylation changes of the target CpG site are subjected to principal component analysis, and the weights of these methylation changes are adjusted using the loadings of the first principal component analysis and then summed to obtain the MIND of the present invention. Detailed instructions for each step are as follows.

### 1. Differential Expression (DE)

First, we generated a cell library consisting of lung cancer patient-derived CAFs and NFs using a primary culture system. To examine cell type purity of the CAF lines via flow cytometry, we performed immunofluorescence staining of 2 CAF markers, CD90 and FAP, and 1 epithelial marker, EPCAM. These CAFs were CD90 positive and FAP positive but EPCAM negative, in contrast with the A549 cancer cells, indicative of no cancer cell contamination. In addition, no EGFR mutation signal was detected in the CAF culture of a patient with somatic tumor cell mutation of EGFR (T>G mutation, L858R) using targeted sequencing, confirming no cancer cell contamination in these CAFs.

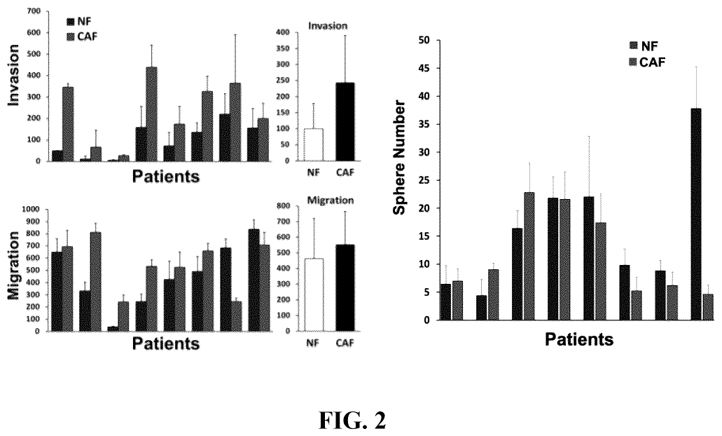

Proceeding with the investigation of phenotypic heterogeneity, we treated lung cancer cells (CLS1) with conditioned medium (CM) from paired CAFs and NFs to examine their contributions to the tumor-promoting ability in cancer migration, invasion, and sphere formation. The results are shown in FIG. 2. The results reflect the considerable diversity of CAFs across patients.

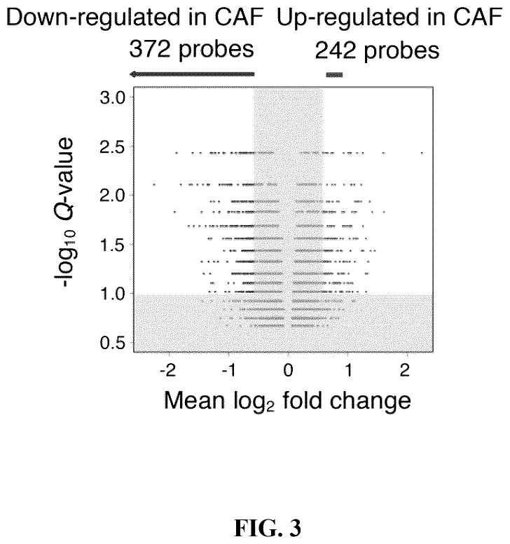

Subsequently, transcriptomic differences between CAFs and NFs were analyzed, that is, differential expression of genes. We profiled gene expression of the CAF and NF pairs from 25 non-small-cell lung cancer (NSCLC) patients using the Affymetrix GeneChip Human Genome U133 Plus 2.0 array. Significance analysis of microarrays (SAM) led to the identification of 614 differentially expressed (DE) probes between CAFs and NFs with a difference in fold change greater than 1.5 at a false discovery rate control Q value of less than 0.1 (not shown in the drawings). The results are shown in FIG. 3. Among 614 differentially expressed (DE) probes, 242 upregulated probes were annotated to 189 genes while 372 downregulated probes were annotated to 272 genes.

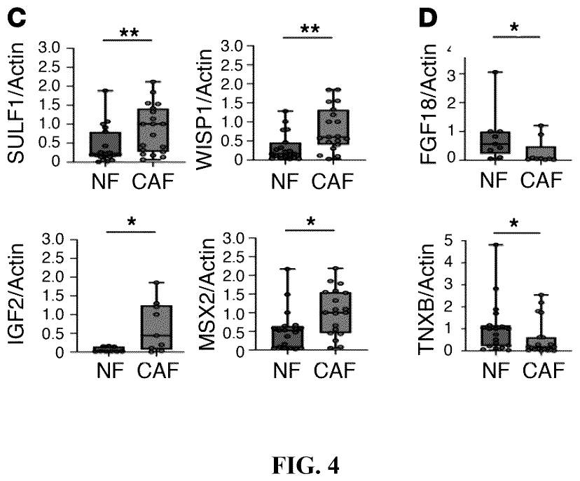

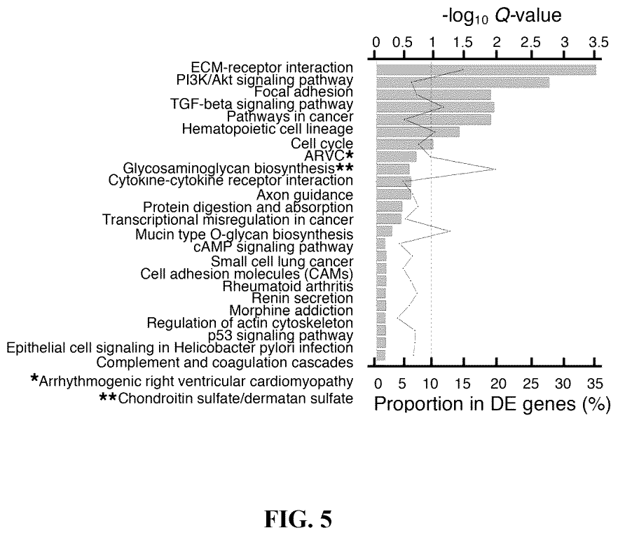

In addition, in DE probes for CAFs and NFs, top DE genes were further analyzed by quantitative real-time PCR (qPCR). The results are shown in FIG. 4, including SULF1, WISP1, IGF2, and MSX2 with upregulated expression, and FGF1 and TNXB with downregulated expression. Furthermore, enrichment analysis was further used to analyze the biological significance of the affected expression in CAFs. The results are shown in FIG. 5. ECM-receptor interaction, PI3K/Akt signaling pathway, focal adhesion, and TGF-β signaling pathway are highly enriched, suggesting a prominent cancer mediator role for CAFs. The top-ranked ECM-receptor interaction pathway featured upregulated CD36 and COL11A1 and downregulated TNC and TNXB, among others. As a component of the TME, the ECM plays a key role in fibroblast activation and phenotypic heterogeneity, and thus its alteration can influence cancer development and progression.

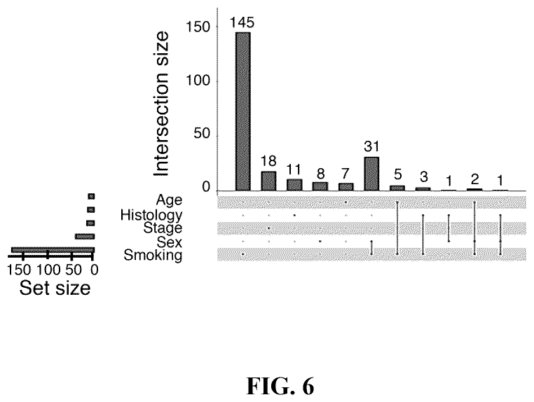

Additional analyses of DE probes were conducted to investigate the correlation with clinical variables. Patients were stratified separately into different groups according to histology, cancer stage, sex, and smoking status. We compared the between-group differences in the fold change of each DE probe to determine the correlated probes, using Welch's t test (P<0.05). For age, we treated it as a continuous variable and used Pearson's correlation coefficient (P<0.05). As shown in FIG. 6, the sizes of intersections among the 5 sets of DE probes correlated with clinical variables were visualized by UpSet plot. Notably, the set of smoking-correlated DE probes overwhelmingly outnumbered other histological variables. Out of the 614 DE probes, 187 (30%) were significantly correlated with smoking status.

### 2. Differential Methylation (DM)

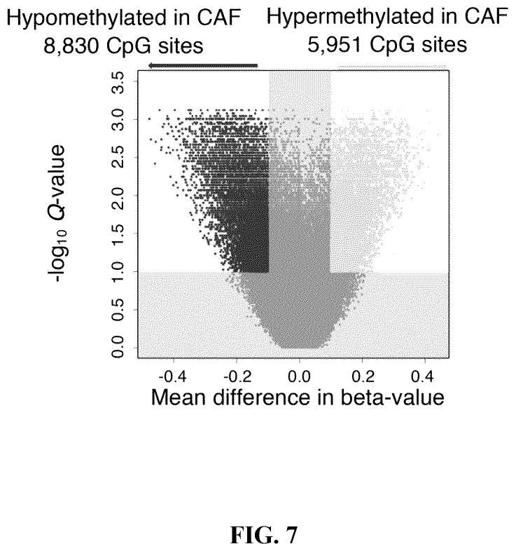

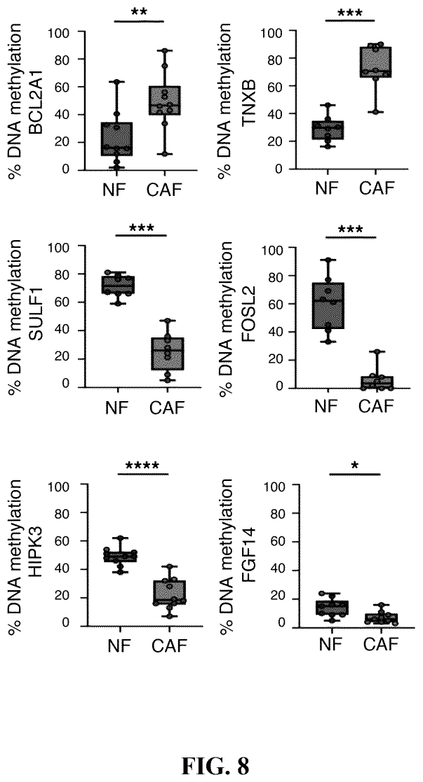

First, we conducted genome-wide DNA methylation profiling on the primary cultured CAF/NF pairs from 26 NSCLC patients using the Infinium Human Methylation 450K array. The β values of CpG sites were found to follow a bimodal distribution (not shown in the drawings). As shown in FIG. 7, requiring the β-value difference (Δβ) to be greater than 0.1, we identified 14,781 differentially methylated (DM) CpG sites between CAFs and NFs with Q values less than 0.1; Among them, 8,830 CpG sites (60%) exhibited hypomethylation, while 5,951 (40%) were hypermethylated in CAFs relative to NFs (not shown in the drawings). Methylation patterns of some NF samples appeared similar to those of CAF samples (not shown in the drawings), rendering them more susceptible to developing premalignancy in these patients. In addition, pyrosequencing was further performed on selected DM CpG sites to quantify methylation differences between CAFs and NFs. The results are shown in FIG. 8, including hypermethylated BCL2A1 and TNXB, and hypomethylated SULF1, FOSL2, HIPK3, and FGF14.

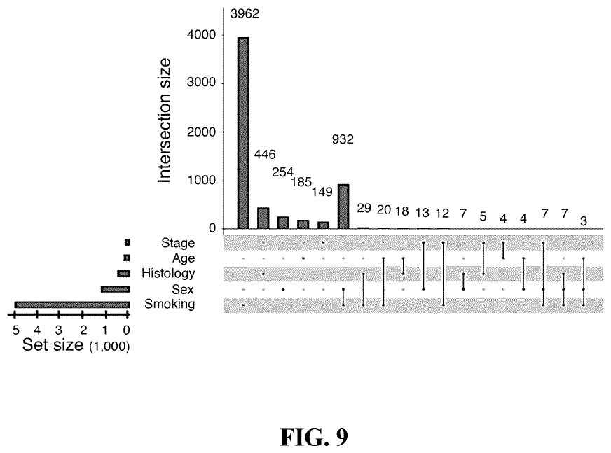

In addition, the correlation between differentially methylated CpG sites and clinical variables was also analyzed. Histology, cancer stage, sex, and smoking status were used to stratify patients. Separately for each clinical variable, we compared the between-group differences in β-value change (Δβ) of each DM CpG site to determine the correlated sites, using Welch's t test (P<0.05). As shown in FIG. 9, the sizes of intersections among the 5 sets of DM sites correlated with clinical variables were visualized by an UpSet plot. Strikingly, consistent with the expression profiling, smoking had a strong correlation among clinical variables. A total of 4,972 DM sites (4,972/14,781, 34%) were significantly correlated with smoking status (never vs. ever; P<0.05). Even after controlling Q less than 0.1, we still retained 3,707 smoking associated DM sites (3,707/14,781, 25%) with larger Δβ in ever-smokers than in never-smokers, while no DM sites were retained for the other 4 clinical variables, suggesting smoking as an important player in the determination of the unique CAF-specific DNA methylation pattern.

### 3. Integration of Differential Expression and Differential Methylation

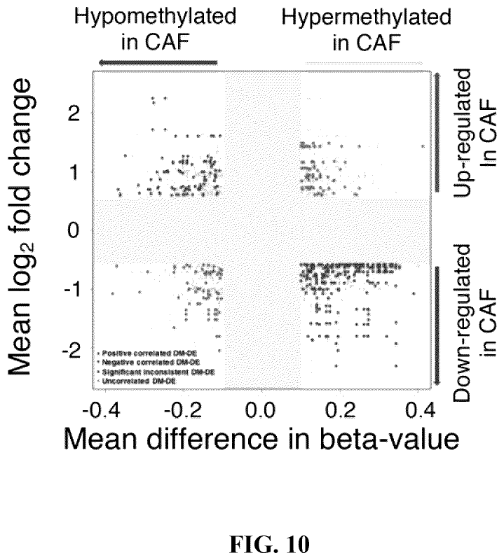

To investigate how DNA methylation at CpG sites of CAF/NF may cis-regulate gene expression, we matched the 614 DE probes obtained earlier with 14,781 DM sites by gene symbol, leading to a total of 1,193 DM-DE pairs. For each DM-DE pair, Spearman's ρ was used to evaluate the correlation between the methylation changes (Δβ) and the expression fold changes. The results are shown in FIG. 10. A total of 482 DM-DE pairs showed significant correlations (Q<0.1) with the signs consistent with the ratios of the mean log 2(foldchange) to the mean Δβ. Among them, 340 (70.5%) pairs were negatively correlated.

Subsequently, these 482 cis-correlated DM-DE pairs were intersected with the aforementioned 3,707 smoking-associated DM CpG sites, and 93 CpG sites were obtained. In order to further select a smaller number of high-quality methylated probes, 10 CpG sites with nearby SNPs were first excluded. Only CpG sites with larger ΔB between CAF and NF were retained, resulting in a total of 54 selected CpG sites. In summary, these 54 differentially methylated CpG sites were screened out with the following four characteristics: (1) patients related to smoking status; (2) related to the expression of target genes; (3) filtration of SNP; and (4) one gene only corresponds to a single CpG site.

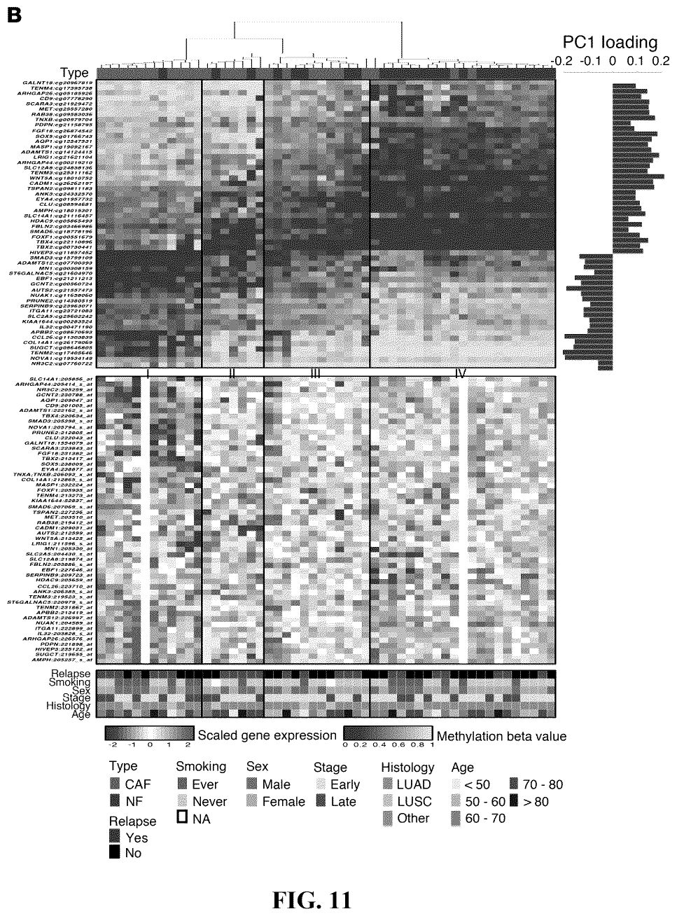

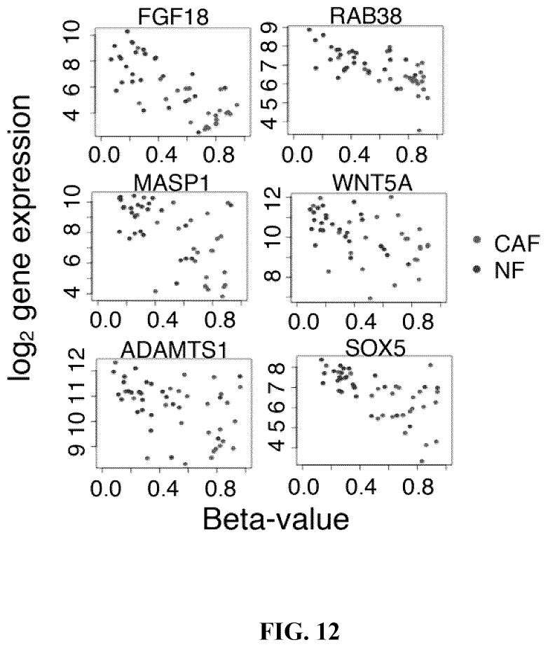

Subsequently, hierarchical DNA methylation clustering of these 54 CpGs was conducted on 52 CAF/NF samples from 26 patients, and 4 subgroups were identified. Group I consisted of CAFs and Group IV mostly consisted of NFs. Notably, we found that certain NFs or CAFs possessed specific methylation patterns, indicating that varied clinical status, such as recurrence, might be encoded. Together with the gene expression profile of the matched 54 genes shown in parallel in FIG. 11, these findings illustrate a distinct pattern of CAFs versus NFs both in methylation and expression. The β value-to-gene expression correlations for 54 genes are shown individually in FIG. 12, in which red represents CAF and blue represents NF.

### 4. Establishment of Distinct Methylation Index for NF/CAF Discrimination (MIND)

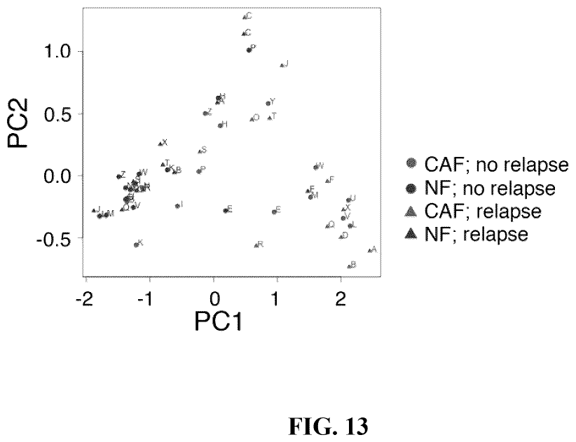

We performed principal component analysis (PCA) on the β values measured at the 54 CpG sites across 26 CAF/NF pairs. The results are shown in FIG. 13, and a separation between CAFs and NFs was visible. Samples from patients with recurrent cancer are represented by triangles and samples from patients without recurrent cancer are represented by circles. The first principal component analysis (PC1) explains 67.0% of the total variance, and the second principal component analysis (PC2) explains 7.5% of the total variance (not shown in the drawings). We then took PC1 as a concise summary of genome-wide methylation profiles and assessed its ability to discriminate CAFs from NFs. More specifically, we constructed a distinct methylation index for NF/CAF discrimination (MIND) of the present invention by simply summing the centered B values of the 54 CpG sites with weights determined by the loadings of PC1, as shown in FIG. 11. The loadings of principal component analysis are the linear combination coefficients of the original variables that constitute the principal component.

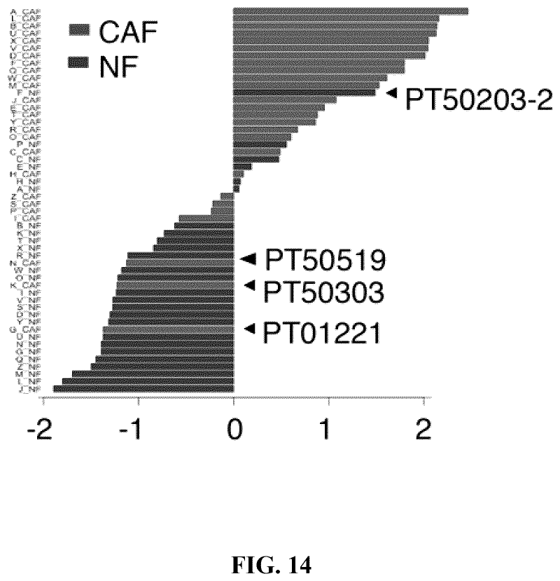

The MIND of the present invention is used to score CAF and NF in the aforementioned 26 patients. To compare the β values of the 54 CpG sites in CAF or NF, the first principal component analysis of PC1 was first performed. The scoring method is MIND=the sum of the β values of the 54 CpG sites in CAF or NF multiplied by their loadings, ordered from the largest value to the smallest value. As shown in FIG. 14, where CAF is in red and N is in blue, the difference between CAF and NF can be clearly seen. Interestingly, further inspection of the NF sample that was mixed with CAFs revealed that this patient had developed recurrence (PT50203-2), indicating suspicious malignancy of the NFs. In contrast, the CAFs that were mixed with NFs came from relapse-free patients (PT50519, PT50303, and PT01221). Our findings suggest that MIND of the present invention may have the potential to robustly detect premalignancy across individual patients.

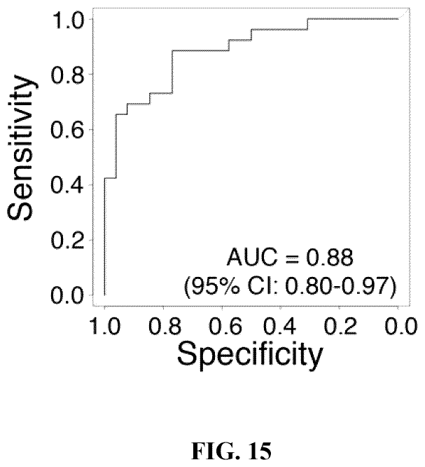

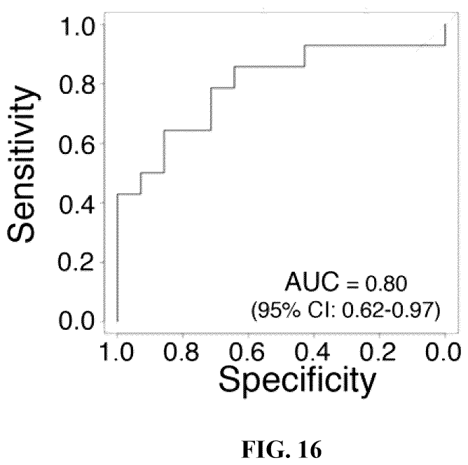

Next, the performance of MIND of the present invention for discriminating CAF from NF was evaluated by receiver operating characteristic curve (ROC). The results are shown in FIG. 15. It can be seen that an AUC of 0.88 (95% CI=0.80-0.97) with 88% sensitivity and 77% specificity at the optimal cutoff by Youden's index. To validate the CAF/NF discriminatory ability of MIND of the present invention, we used a validation cohort of paired CAFs/NFs cultured from 14 NSCLC patients, and further analysis was performed with ROC curve. The results are shown in FIG. 16, yielding an AUC of 0.80 (95% CI=0.62-0.97). Another validation cohort used was a public data set with paired CAFs/NFs from 12 NSCLC patients (GSE68851) and MIND of the present invention also yielded good discrimination (AUC=0.83, 95% CI=0.66-1.0, 73% sensitivity and 92% specificity) (not shown in the drawings). These results show that MIND of the present invention can be effectively used to discriminate CAF from NF.

Furthermore, we also performed PCA on the mRNA expression profile of the same 54 genes and produced a gene expression index (MIND-GE), which is the sum of the standardized expression with weights determined by the loadings of PC1. MIND-GE also showed high power in distinguishing CAFs from NFs (AUC=0.83, 95% CI=0.70-0.95), and this finding was consistent in another independent cohort of CAF/NF pairs from 15 NSCLC patients (GSE22874) (AUC=0.93, 95% CI=0.84-1.0).

### Example 2 the Potential of MIND of Present Invention in Quantifying TME Malignancy

In embodiments of the present invention, to explore whether MIND of the present invention could serve as a useful summary of the CAF methylome for grading the degree of TME malignancy across individual patients, we performed phenotypic studies. CAFs were selected from the aforementioned 26 CAFs, and the first principal component analysis was performed to obtain the loading value of PC1 (see FIG. 14). Each β value was multiplied by the loading value found in FIG. 14 to obtain MIND scores according to the MIND definition to evaluate how cancer cells may react differently to the heterogeneous cell culture environment contributed by CAFs with different MIND scores. We collected conditioned medium (CM) from the CAFs with higher (MIND^{high}) or lower (MIND^{low}) MIND scores and applied them to 2 lung cancer cell lines (A549 and CL1-0). Assays of cell viability and invasion were performed to better clarify the objective and potential impact of MIND of the present invention. The results showed that both the viability and invasive abilities of the cancer cells were highly promoted with the treatment of CM from MIND^{high} CAFs compared with that from the MIND^{low} CAFs, suggesting that the malignancy level of CAFs can be quantitated effectively via MIND of the present invention.

According to the seed and soil theory, a poor-graded TME (like a bad soil) leads to the poor survival of a patient; therefore, in the present invention, we used an objective way to separate the patients of our discovery cohort into a poor-TME group and a good-TME group by MIND of the present invention (without the use of patient survival data). We computed the cutoff point for MIND of the present invention to separate CAFs from NFs by controlling the probability of misclassifying NFs at no more than 5% while maximizing the probability of correct classification of CAFs. Using this cutoff point, we assigned the patients with CAF MIND scores that were higher than the cutoff point (MIND^{high}) to the poor-TME group (determining to be a TME with high malignant potential) and kept the rest (MIND^{low}) in the good-TME group (determining to be a TME with low malignant potential).

### Example 3 Prognostic Performance of MIND of Present Invention

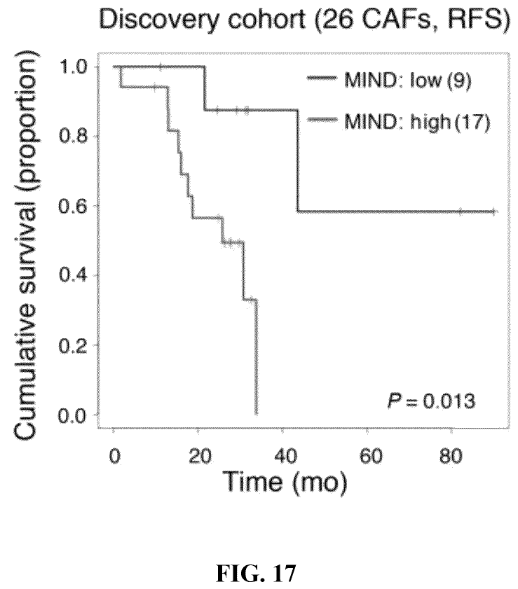

In embodiments of the present invention, the ability of MIND of the present invention was further evaluated to evaluate patient prognosis. Using the longitudinal patients' follow-ups of tumor recurrence in the discovery cohort, we compared the relapse-free survival (RFS) curves of the poor-TME and good-TME groups, stratified by the objective split described above, to assess the clinical significance of MIND. The results are shown in FIG. 17. The result showed that MIND^{high} patients had poor outcomes (P=0.013, log-rank test). Multivariate Cox regression confirmed the significance of MIND adjusted by cancer stage, age, sex, and smoking status (HR=9.29, 95% CI=1.14-75.44, P=0.037; Table 1).

TABLE 1

| | Discovery cohort [symbol] | | | | | |
|---|---|---|---|---|---|---|
| | [symbol] | | | [symbol] | | |
| Variable | HR | 95% CI | P | HR | 95% CI | P |
| MIND | 9.35 | 1.15-75.86 | 0.036 | 9.29 | 1.14-75.44 | 0.037 |
| Stage | 1.58 | 0.44-5.69 | 0.484 | — | — | — |
| Age | 1.03 | 0 [symbol] -1.0 [symbol] | 0.214 | — | — | — |
| Sex | 0.94 | 0.30-2.98 | 0.917 | — | — | — |
| Smoking | 1.89 | 0.55- [symbol] 5 [symbol] | 0.315 | — | — | — |

| | Validation cohort [symbol] | | | | | |
|---|---|---|---|---|---|---|
| | [symbol] | | | [symbol] | | |
| Variable | HR | 95% CI | P | HR | 95% CI | P |
| MIND | 15.15 | 1.8- [symbol] | 0.009 | 29.97 | 2.19-6 [symbol] | 0.006 |
| Stage | 6.52 | 1. [symbol] -66.22 | 0.032 | 13.5 | 1.41-1 [symbol] | 0.021 |
| Age | 0.97 | 0.86-1.09 | 0. [symbol] 28 | — | — | — |
| Sex | 1.81 | 0.35-10.96 | 0.471 | — | — | — |
| Smoking | 2.63 | 0.5 [symbol] - [symbol] | 0.243 | — | — | — |

| | [symbol] | | | | | |
|---|---|---|---|---|---|---|
| | [symbol] | | | [symbol] | | |
| Variable | HR | 95% CI | P | HR | 95% CI | P |
| MIND | 1.83 | 1.22-2.75 | 0.004 | 2.00 | 1.32-3.04 | 0.001 |
| Stage | 4.20 | 2.63-6.71 | <0.001 | 4.40 | 2.74-7.08 | <0.001 |
| Age | 0.98 | 0.97-1.0 [symbol] | 0.0 [symbol] | 0.98 | 0.96-1.00 | 0.056 |
| Sex | 1.40 | 0.93-2.11 | 0.108 | 1.6 | 1.05-2.41 | 0.027 |
| Smoking | 1.05 | 0.57-1.92 | 0. [symbol] | — | — | — |
| | [symbol] | | | | | |
| | [symbol] | | | [symbol] | | |
| Variable | HR | 95% CI | P | HR | 95% CI | P |
| MIND | 1.46 | 1.07-2.01 | 0.01 [symbol] | 1.42 | 1.02-1. [symbol] 7 | 0.037 |
| Stage | 2.45 | 1.74-3.41 | <0.001 | 2.27 | 1.60-3.22 | <0.001 |
| Age | 1.01 | 0.39-1.02 | 0.412 | — | — | — |
| Sex | 1.06 | 0.78-1.45 | 0.707 | — | — | — |
| Smoking | 1.01 | 0.54-1.59 | 0. [symbol] | — | — | — |

[symbol]

penalized likelihood method was used for this small data set.
n, number of patients with nonmissing value of MIND and survival data; RFS, relapse-free survival, OS, overall survival, HR, hazard ratio; CI, confidence interval. [symbol]
indicates data missing or illegible when filed

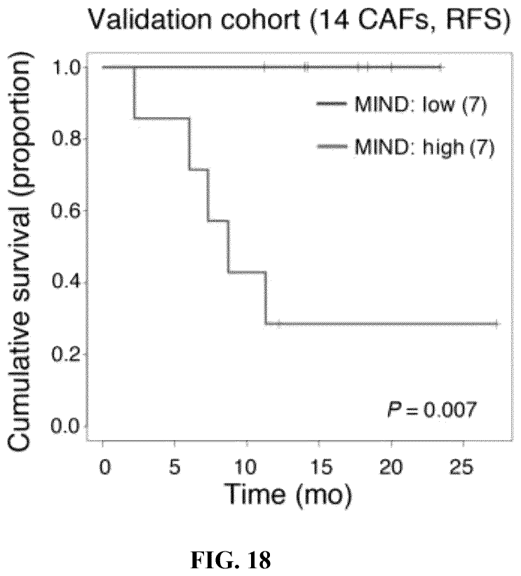

We also applied MIND of the present invention to our validation cohort of 14 patients. We ranked patients according to the MIND scores of their CAFs and split them into 2 groups of equal size. RFS was analyzed by log-rank test (P=0.007; FIG. 18). Multivariate Cox regression further confirmed the prognostic ability of MIND of the present invention adjusted by clinical variables (HR=29.17, 95% CI=2.19-6,520.53, P=0.006; Table 1).

In addition, although GSE39279 and The Cancer Genome Atlas Lung Adenocarcinoma (TCGA-LUAD), have methylome data on the tumors, instead of CAFs, due to the impurity of tumor samples, some CAF content may be retained in the resected tumor tissue and profiled together with the bulk of tumor cells. Although the signals from CAFs would have deteriorated and the prognostic performance of MIND could be compromised, we still applied MIND to the 2 cohorts.

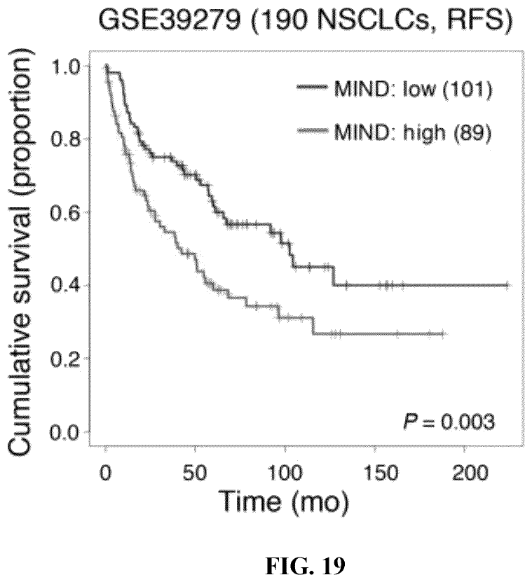

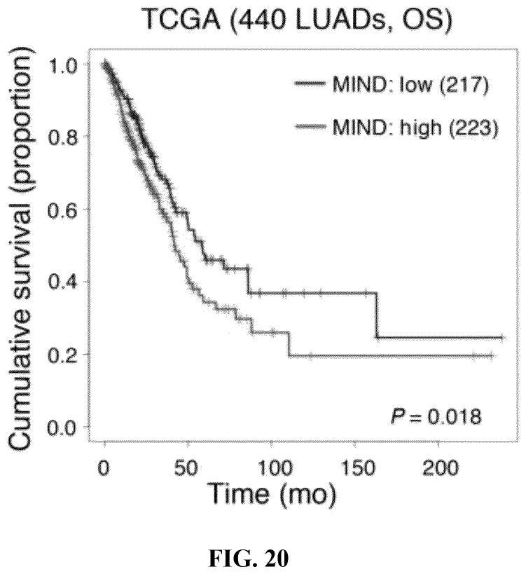

First, for each patient, each β value is multiplied by the loading value found in FIG. 14 to calculate the MIND score. With the median cutoff, patients were split evenly into 2 groups (MIND^{high} vs. MIND^{low}) without the input of survival data. After that, those with no survival data were removed from further survival analysis. The results are shown in FIGS. 19 and 20. Promisingly, the results showed that patients with high MIND scores had significantly shorter RFS (P=0.003 in GSE39279) and shorter overall survival (OS) (P=0.018 in TCGA-LUAD) than those with low MIND scores. This finding was further confirmed by univariate and multivariate Cox regression (Table 1). Conclusively, the MIND index of the present invention could be used to define high-risk populations for early detection of recurrence. It has the potential to detect premalignant TMEs in patients with different clinical status.

In addition to DNA methylation, MIND-GE also showed significant performance in recurrence prediction in our discovery cohort using the median as cutoff (P=0.041). Applying MIND-GE to the gene expression profiling data of the TCGA-LUAD cohort found P=0.069 in OS prediction. The result showed that MIND of the present invention outperformed MIND-GE in the prognostic assessment.

In summary, the present invention has established index for evaluating malignancy level of tissue microenvironment and predicting tumor recurrence and precancerous lesions. A methylation index that summarizes the β values of these CpGs was built for NF/CAF discrimination (MIND) with high sensitivity and specificity. The potential of MIND in detecting premalignancy across individual patients was shown in the present invention. MIND succeeded in predicting tumor recurrence in multiple lung cancer cohorts without reliance on patient survival data, suggesting that the malignancy level of TME may be effectively graded by this index. Precision TME grading may provide additional pathological information to guide cancer prognosis and open up more options in personalized medicine.

What is claimed is:

1. A method of establishing index for evaluating malignancy level of tissue microenvironment, comprising:
(a) analyzing cancer-associated fibroblasts and normal fibroblasts from a cancer patient to obtain differentially methylated CpG sites and differentially expressed genes;
(b) overlapping the differentially methylated CpG sites with the differentially expressed genes to obtain a differentially methylated-differentially expressed pair;
(c) analyzing correlation between DNA methylation change value and gene expression fold change in the differentially methylated-differentially expressed pair to obtain a differentially methylated site with cis-correlation;
(d) analyzing another differentially methylated site associated with clinical characteristics of the cancer patient and intersecting with the differentially methylated site to obtain a plurality of target sites;
(e) performing principal component analysis on a group consisting of methylation β values of the plurality of target sites;
(f) obtaining a loading value of a first principal component analysis of the methylation β values corresponding to each of the plurality of target sites; and
(g) multiplying the methylation β values of the plurality of target sites by the loading value and summed to establish the index for evaluating malignancy level of tissue microenvironment.

2. The method of claim 1, wherein the cancer patient is a lung cancer patient.

3. The method of claim 2, wherein one of the clinical characteristics is smoking.

4. A method for evaluating malignancy level of tissue microenvironment, comprising:
(a) determining a methylation β value of a target cell at a plurality of target sites in an in vitro sample of a subject;
(b) multiplying the methylation β value of the plurality of target sites by a loading value of corresponding target site in the subject and summed to obtain an index score for evaluating malignancy level of tissue microenvironment; and
(c) determining the tissue microenvironment in the subject to have potential to promote tumor malignancy when the index score is higher than a cutoff point;
wherein the loading value of corresponding target site in the subject is a loading value of a first principal component analysis obtained by performing principal component analysis on a group, wherein the group consists of the methylation β value of the plurality of target sites in the target cell of a subject suffering from tumor.

5. The method of claim 4, wherein the potential to promote tumor malignancy is potential to produce precancerous lesions.

6. The method of claim 4, wherein the potential to promote tumor malignancy is potential to promote cancer recurrence.

7. The method of claim 4, wherein the subject suffering from tumor is a subject suffering from lung cancer.

8. A system for evaluating malignancy level of tissue microenvironment, comprising:
a detection module configured to determine and obtain a methylation β value of a target cell at a plurality of target sites in an in vitro sample of a subject;
an analyzing and processing module configured to multiply the methylation β value of the plurality of target sites by a loading value of corresponding target site in the subject and summed to obtain an index score for evaluating malignancy level of tissue microenvironment and predicting tumor recurrence and precancerous lesions; and
an information comparison module configured to compare whether the index score is higher than a cutoff point to determine whether the tissue microenvironment in the subject has or does not have potential to promote tumor malignancy.

9. The system of claim 8, wherein the potential to promote tumor malignancy is potential to produce precancerous lesions.

10. The system of claim 8, wherein the potential to promote tumor malignancy is potential to promote cancer recurrence.
